(19) United States
(12) Patent Application Publication
(10) Pub. No.: US 2012/0027681 A1
(43) Pub. Date: Feb. 2, 2012

(54) Low-Aspect Ratio Carbon Nanostructures

(75) Inventors: Yung Joon Jung, Lexington, MA (US); Hyunkyung Chun, Malden, MA (US); Latika Menon, Boston, MA (US)

(73) Assignee: NORTHEASTERN UNIVERSITY, Boston, MA (US)

(21) Appl. No.: 13/255,697

(22) PCT Filed: Mar. 11, 2010
(86) PCT No.: PCT/US2010/026986

§ 371 Date: Oct. 20, 2011

Related U.S. Application Data
(60) Provisional application No. 61/159,289, filed on Mar. 11, 2009.

(51) Int. Cl. A61K 49/00; A61K 9/00; A61K 33/00; B05D 5/00; H01J 37/31; B32B 1/00; C23C 16/26; B82Y 5/00; B82Y 40/00

(52) U.S. Cl. 424/9.1; 428/34.1; 424/400; 424/600; 427/249.6; 264/485; 977/842; 977/906

(57) ABSTRACT

Low-aspect ratio nanostructures, such as nanocups, nanorings, and arrays of nanocups and nanorings, methods of fabrication of nanostructures, and methods of using nanostructures are disclosed.

### CROSS-REFERENCE TO RELATED APPLICATIONS

This application claims the benefit of priority to U.S. Provisional Application No. 61/159,289, filed Mar. 11, 2009, the contents of which are incorporated by reference herein in their entirety.

### FIELD OF THE INVENTION

The invention is in the field of nanotechnology including nano-medicine and nano-metrology.

### BACKGROUND OF THE INVENTION

Short structures, including very short nanotubes (Lu et al. (1996) Carbon 34:814-816; Shelimov et al. (1998) Chem. Phys. Lett. 282:429-434; Liu et al. (1998) Science 280:1253-1256; Liesbeth et al. (1997) Appl. Phys. Lett. 71:2629-2631) have been impossible to grow by existing techniques due to the difficulty in controlling and terminating growth during initial stages.

Currently, short carbon nanotubes (“CNTs”) are fabricated by cutting longer CNTs (micron scale) using acid based chemical treatments. Such chemically shortened CNTs have many disadvantages. For example, it is very difficult to make very low L/D ratio (it has been report only down to 10, L/D ratio) and the length of such CNTs is not uniform. Additionally, it is difficult to control the L/D ratio, diameter, and length of such nanotube structures. Also, low-dimensional graphitic structures with larger diameter (more than 30 nm) have not thus far been made.

Various groups have explored the potential of using unique hollow structures from graphitic carbon, such as carbon nanotubes, for building multifunctional nanostructures (Meng et al. (2005) Proc. of Nat'l. Acad. Sci. U.S.A. 102:7074-7078; Martin (1994) Science 266:1961-1966; Davydov et al. (1999) J. Appl. Phys. 86:3983-3987; Sui et al. (2002) Thin Solid Films 406:64-69; Allen et al. (2008) ACS Nano 2:1914-1920) useful in a large number of applications (Weda et al. (2008) Polymer 49:1467-1474;Ye et al. (2007) Carbon 45:315-320; Bai et al. (2001) Appl. Phys. Lett. 79:1552; Zhong et al. (2001) Appl. Phys. Lett. 79:3500; Ma et al. (1999) Appl. Phys. Lett. 75:3105; Broz et al. (2006) Nano Lett. 6:2349-2353). However, current technologies are significantly limited by the difficulty of tailoring morphology and aspect ratio of individual nanoscale units.

Thus, what is needed are low aspect, hollow carbon nanostructures and methods of making them.

### SUMMARY OF THE INVENTION

The invention is based, at least in part, on the discovery of a process for fabricating low-aspect ratio nanostructures. This discovery was exploited to develop the invention, which, in one aspect, features a hollow, low-aspect ratio nanostructure having a length, a transverse diameter, and a continuous lateral wall defining an interior space.

In some embodiments, the nanostructure has an aspect ratio of about 0.5, about 1, about 1.5, about 2, about 2.5, about 3, about 3.5, about 4, about 4.5, about 5, about 6, about 7, about 8, about 9, or about 10. In other embodiments, the nanostructure has an aspect ratio of about 0.5 to about 10, about 1 to about 5, about 1.5 to about 3, about 0.5 to about 1, about 1.5 to about 2, about 2 to about 3, about 3 to about 5, or about 6 to about 10.

In other embodiments, the length of the nanostructure is about 20 nm, about 30 nm, about 40 nm, about 50 nm, about 60 nm, about 70 nm, about 80 nm, about 90 nm, about 100 nm, about 150 nm, about 200 nm, about 250 nm, about 300 nm, about 400 nm, about 500 nm, about 600 nm, about 700 nm, about 800 nm, about 900 nm, or about 1,000 nm. In yet other embodiments, the length of the nanostructure is about 20 nm to about 1,000 nm, about 40 nm to about 500 nm, about 60 nm to about 250 nm, about 80 nm to about 200 nm, about 20 nm to about 40 nm, about 50 nm to about 80 nm, about 100 nm to about 500 nm, or about 600 nm to about 1,000 nm.

In some embodiments, the transverse diameter of the nanostructure is about 10 nm, about 20 nm, about 30 nm, about 40 nm, about 50 nm, about 60 nm, about 70 nm, about 80 nm, about 90 nm, about 100 nm, about 150 nm, about 200 nm, about 250 nm, about 300 nm, about 400 nm, or about 500 nm. In other embodiments, the transverse diameter is about 10 nm to about 500 nm, about 20 nm to about 250 nm, about 40 nm to about 150 nm, about 10 nm to about 40 nm, about 50 nm to about 100 nm, about 110 nm to about 150 nm, about 160 nm to about 250 nm, or about 260 nm to about 500 nm.

In some embodiments, the lateral wall has a thickness of about 1 nm, about 2 nm, about 3 nm, about 4 nm, about 5 nm, about 6 nm, about 7 nm, about 8 nm, about 9 nm, about 10 nm, about 15 nm, about 20 nm, about 25 nm, about 30 nm, about 35 nm, about 40 nm, about 50 nm, about 60 nm, about 70 nm, about 80 nm, about 90 nm, about 100 nm, about 150 nm, about 200 nm, or greater. In yet other embodiments, the lateral wall has a thickness of about 1 nm to about 200 nm, about 10 nm to about 100 nm, about 20 nm to about 50 nm, about 1 nm to about 5 nm, about 6 nm to about 10 nm, about 15 nm to about 50 nm, about 60 nm to about 100 nm, about 100 nm to about 150 nm, or about 150 nm to about 200 nm.

In other embodiments, the lateral wall defines an enclosed space of about 30 nm³, about 50 nm³, about 75 nm³, about 100 nm³, about 150 nm³, about 200 nm³, about 250 nm³, about 300 nm³, about 400 nm³, or about 500 nm³.

In some embodiments, the nanostructure comprises carbon or silica.

In yet other embodiments, the nanostructure is a nanocup having an open top and a closed bottom. In some embodiments, the nanocup further comprises a lid. In other embodiments, the nanostructure is a nanoring having an open top and an open bottom.

In other embodiments, the nanostructure further comprises one or more agents within the interior space of the nanostructure. In some embodiments, the agent is a therapeutic agent described herein. In some embodiments, the agent is a detection agent described herein. In yet other embodiments, the nanostructure further comprises one or more therapeutic agent and one or more detection agents. In particular embodiments, the agent is hydrogen. In other embodiments, the agent is a metal or polymer.

In yet other embodiments, the nanostructure further comprises one or more agents attached to the nanostructure. In some embodiments, the agent is attached to an outer surface of the lateral wall, opposite the interior space. In other embodiments, the agent is attached to the surface of the lateral wall facing the interior space.

In another aspect, the invention features an array comprising a plurality of nanostructures described herein. In one embodiment, the array comprises a plurality of nanocups. In some embodiments, the array comprises about 2, about 5, about 10, about 20, about 30, about 40, about 50, about 60, about 70, about 80, about 90, about 100, about 200, about 400, about 500, about 1,000, or more nanocups. In other embodiments, the array comprises about 2 to about 1,000, about 100 to about 1,000, about 500 to about 10,000, about 1,000 to about 10,000, about 100 to about 500, about 500 to about 1,000, about 1,000 to about 5,000, or about 5,000 to about 10,000 nanocups.

In some embodiments, the array comprises a support layer that contacts the lateral walls of adjacent nanostructures. In certain embodiments, the support layer is flexible. In some embodiments, the support layer comprises a graphite layer. In particular embodiments, the support layer has a thickness of about 5 nm to about 200 nm.

In other embodiments, the lengths and/or transverse diameters of the nanostructures are uniform. In certain embodiments, about 10%, about 20%, about 30%, about 40%, about 50%, about 60%, about 70%, about 80%, about 90% or 100% of the nanostructures have the same length and/or transverse diameter. In other embodiments, about 10% to about 20%, about 30% to about 40%, about 50% to about 60%, about 70% to about 80%, or about 90% to about 100% of the nanostructures have the same length and/or transverse diameter.

In some embodiments, the array is a flexible sheet comprising a plurality of nanostructures.

In another aspect, the invention features a method of forming an array of a plurality of hollow, low-aspect ratio nanostructures, the method comprising preparing an anodized aluminum oxide (AAO) template comprising a plurality of nanochannels by two-step anodization; and disposing a graphitic carbon layer onto the AAO template, thereby producing an array of hollow, low-aspect ratio nanostructures.

In some embodiments, the graphitic carbon layer is disposed onto the AAO template by chemical vapor deposition.

In some embodiments, each nanochannel has a length, a transverse diameter, and a lateral wall defining an interior space.

In some embodiments, the nanochannel has an aspect ratio of about 0.5, about 1, about 1.5, about 2, about 2.5, about 3, about 3.5, about 4, about 4.5, about 5, about 6, about 7, about 8, about 9, or about 10. In other embodiments, the nanochannel has an aspect ratio of about 0.5 to about 10, about 1 to about 5, about 1.5 to about 3, about 0.5 to about 1, about 1.5 to about 2, about 2 to about 3, about 3 to about 5, or about 6 to about 10.

In other embodiments, the length of the nanochannel is about 20 nm, about 30 nm, about 40 nm, about 50 nm, about 60 nm, about 70 nm, about 80 nm, about 90 nm, about 100 nm, about 150 nm, about 200 nm, about 250 nm, about 300 nm, about 400 nm, about 500 nm, about 600 nm, about 700 nm, about 800 nm, about 900 nm, or about 1,000 nm. In yet other embodiments, the length of the nanochannel is about 20 nm to about 1,000 nm, about 40 nm to about 500 nm, about 60 nm to about 250 nm, about 80 nm to about 200 nm, about 20 nm to about 40 nm, about 50 nm to about 80 nm, about 100 nm to about 500 nm, or about 600 nm to about 1,000 nm.

In some embodiments, the transverse diameter of the nanochannel is about 10 nm, about 20 nm, about 30 nm, about 40 nm, about 50 nm, about 60 nm, about 70 nm, about 80 nm, about 90 nm, about 100 nm, about 150 nm, about 200 nm, about 250 nm, about 300 nm, about 400 nm, or about 500 nm. In other embodiments, the transverse diameter is about 10 nm to about 500 nm, about 20 nm to about 250 nm, about 40 nm to about 150 nm, about 10 nm to about 40 nm, about 50 nm to about 100 nm, about 110 nm to about 150 nm, about 160 nm to about 250 nm, or about 260 nm to about 500 nm.

In some embodiments, the method further comprises modifying one or more nanostructures within the array. In some embodiments, the nanostructure is modified by inserting one or more agents into the interior space of the nanostructure. In some embodiments, the agent is a therapeutic agent described herein. In some embodiments, the agent is a detection agent described herein. In yet other embodiments, the nanostructure further comprises one or more therapeutic agent and one or more detection agents. In particular embodiments, the agent is hydrogen. In other embodiments, the agent is a metal or polymer.

In yet other embodiments, the nanostructure is modified by attaching one or more agents to the nanostructure. In some embodiments, the agent is attached to an outer surface of the lateral wall, opposite the interior space. In other embodiments, the agent is attached to the surface of the lateral wall facing the interior space.

In some embodiments, the nanostructure is a nanocup, and the method further comprises attaching a lid over the open top, resulting in a sealed nanostructure.

In some embodiments, the method further comprises removing the template from the array. In certain embodiments, the template is removed using hydrofluoric acid.

In another aspect, the invention features a method of forming a hollow, low-aspect ratio nanostructure, the method comprising preparing an anodized aluminum oxide (AAO) template comprising a plurality of nanochannels by two-step anodization; disposing a graphitic carbon layer onto the AAO template to form an array of hollow, low-aspect ratio nanostructures; removing the array from the template; and separating one or more nanostructures from the array. In some embodiments, the array is an array of nanostructures described herein.

In some embodiments, the method further comprises increasing the transverse diameters of the nanochannels by treating the template with sulfuric acid or phosphoric acid.

In some embodiments, the separating step comprises inert gas ion milling. In particular embodiments, the inert gas is Ar, He, or N.

In another aspect, the invention features a method of delivering a therapeutic or detection agent to a target cell, the method comprising: providing a nanostructure described herein; modifying the nanostructure with the therapeutic or detection agent; and administering the modified nanostructure to a subject, thereby delivering the agent to the target cell.

In some embodiments, the method further comprises linking a targeting agent to the nanostructure. In particular embodiments, the targeting agent is a nucleic acid, a polypeptide, a polysaccharide, or a small molecule.

In some embodiments, the subject is a vertebrate. In certain embodiments, the subject is a mammal In particular embodiments, the subject is a human.

In another aspect, the invention features a nanostructure produced by any method described herein.

In another aspect, the invention features the use of a nanostructure according to any of the aspects described herein, for the treatment of a disease or a disorder described herein.

The following figures are presented for the purpose of illustration only, and are not intended to be limiting.

### BRIEF DESCRIPTION OF THE DRAWINGS

FIGS. 1A-1D are schematic illustrations of a method for fabricating a template.

FIG. 2A are schematic illustrations of a method for fabricating arrays of nanocups.

FIG. 2B are schematic illustrations of a method for fabricating separated nanocups and separated nanorings.

FIGS. 3A-3E are schematic illustrations of exemplary nanostructures.

FIGS. 4A and 4B are schematic illustrations of exemplary nanostructures for drug delivery.

FIGS. 5A-5C are representations of scanning electron micrographs (SEM) of highly controlled, short, AAO microchannels having a (a) 70 nm length (anodized at 40 V for 25 sec); (b) 200 nm length (anodized at 45 BV for 35 sec); and (c) 400 nm length (anodized at 40 V for 120 sec, where all scale bars are 100 nm.

FIG. 5D is a graphic representation showing the length of nanochannels as a function of a second anodizing time at 45 V.

FIGS. 6A-6B are representations of SEMs showing (6A) nanocups connected with a graphitic layer and where polycrystalline graphite carbon was deposited on both inner and outer surfaces of AAO nanochannels; and (6B) at low magnification, the bottom view of a two-dimensional, planar nanocup based structure, where the scale bars are 100 nm.

FIG. 6C is a representation of a transmission electron micrograph (TEM) showing top, tilted, and side views of connected nanocups due to the flexible nature of the 2-D nanocup-based film, where the scale bar is 100 nm.

FIGS. 7A-7C are representations of SEMs of a two-dimensional carbon nanocup film structure after removing the AAO template. SEM images show (7A) the bottom of highly dense carbon nanocup arrays connected with a thin graphite layer; (7B) a two dimensional and flexible film of carbon nanocups; and (7C) the side view of carbon nanocups (100 nm diameter and 200 nm length) connected with a graphitic layer of 10 nm thicknesses and where scale bars are 200 nm.

FIG. 7D is a representation of a TEM of a two-dimensional carbon nanocup film structure after removing the anodized aluminum oxide (“AAO”) template, where there are connected arrays of carbon nanocup film with 80 nm diameter and 80 nm length and where the scale bar is 50 nm.

FIGS. 8A-8D are representations of SEM images showing architectures of individual carbon nanocup and nanoring structures fabricated using an Ar ion milling process on the connected arrays of carbon nanocup film, where (8A) shows nanocups with the L/D aspect ratio of 3; (8B) shows nanocups with the L/D aspect ratio of 1; (8C) shows multilayered carbon nanoring arrays; and (8D) shows single layered nanoring arrays, and where scale bars are 100 nm.

FIGS. 8E-8F are representations of TEM images from tailored carbon nanostructures revealing (8E) nanoscale cup; and (8F) ring morphology, and where scale bars are 100 nm and 50 nm, respectively.

FIG. 8G is a representation of Micro-Raman spectra (using 532 nm wavelength laser probe) taken from MWNTs (10 μm length), long nanocups (180 nm length), short nanocups (60 nm length), long nanorings (60 nm length), and short nanorings (40 nm length).

FIG. 8H is a graphic representation of a histogram of the intensity ratio between the D band and G band (I_{D}/I_{G}) of each structure. I_{D}/I_{G} is increased from 0.34 to 0.81 as the structures change from MWNTs to short nanorings.

FIG. 9A is a representation of an optical image of a connected carbon nanocup with a water droplet before Ar ion irradiation.

FIG. 9B is a representation of an optical image of a connected carbon nanocup with a water droplet after Ar ion irradiation.

FIGS. 10A-10D are representations of SEM images and FIGS. 10E-10F are representations of TEM images of various carbon nanocup based heterostructures, where ordered arrays of gold nanoparticles were formed selectively inside pores of both (10A) carbon nanocup film structure and (10B) individual nanocup and nanoring structures, and the size of metal nanoparticles inside of nanocup structures can be controlled by adjusting the thickness of a deposited metal film. (10C) Lead nanoparticles (10 nm-15 nm diameters) formed directly from the lead film with 30 nm thickness during thermal annealing process. (10D) A single lead nanoparticle (70 nm-80 nm diameters) inserted from the lead film with 60 nm thickness during a thermal annealing process. TEM images of gold inserted (10E) carbon nanocup films, and (10F) fully separated individual nanocups. All scale bars are 100 nm.

### DETAILED DESCRIPTION OF THE INVENTION

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein, including GenBank database sequences, are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### Definitions

As used herein, a “subject” is a mammal, e.g., a human, mouse, rat, guinea pig, dog, cat, horse, cow, pig, or non-human primate, such as a monkey, chimpanzee, baboon or rhesus.

As used herein, the term “biodegradable” refers to a substance that is decomposed (e.g., chemically or enzymatically) or broken down in component molecules by natural biological processes (e.g., in vertebrate animals such as humans).

As used herein, the term “biocompatible” refers to a substance that has no unintended toxic, blocking, retarding, or other injurious effects on biological functions in a target organism.

As used herein, the term “nanostructure” refers to a low-aspect ratio structure with dimensions on the order of about 1 nm to about 1 μm. Exemplary nanostructures have an aspect ratio of about 0.5 to about 10.

As used herein, the term “nanocup” refers to a low-aspect ratio nanostructure having a general cup shape and open at one end.

As used herein, the term “nanoring” refers to a low-aspect ratio nanostructure having an annular shape and open at two ends.

The term “targeting agent” refers to a ligand or molecule capable of specifically or selectively (i.e., non-randomly) binding or hybridizing to, or otherwise interacting with, a desired target molecule. Examples of targeting agents include, but are not limited to, nucleic acid molecules (e.g., RNA and DNA, including ligand-binding RNA molecules such as aptamers, antisense, or ribozymes), polypeptides (e.g., antigen binding proteins, receptor ligands, signal peptides, and hydrophobic membrane spanning domains), antibodies (and portions thereof), organic molecules (e.g., biotin, carbohydrates, and glycoproteins), and inorganic molecules (e.g., vitamins). A nanostructure described herein can have affixed thereto one or more of a variety of such targeting agents.

As used herein, “about” means a numeric value having a range of ±10% around the cited value.

As used herein, “treat,” “treating” or “treatment” refers to administering a therapy in an amount, manner (e.g., schedule of administration), and/or mode (e.g., route of administration), effective to improve a disorder (e.g., a disorder described herein) or a symptom thereof, or to prevent or slow the progression of a disorder (e.g., a disorder described herein) or a symptom thereof This can be evidenced by, e.g., an improvement in a parameter associated with a disorder or a symptom thereof, e.g., to a statistically significant degree or to a degree detectable to one skilled in the art. An effective amount, manner, or mode can vary depending on the subject and may be tailored to the subject. By preventing or slowing progression of a disorder or a symptom thereof, a treatment can prevent or slow deterioration resulting from a disorder or a symptom thereof in an affected or diagnosed subject.

The term “polymer,” as used herein, refers to a molecule composed of repeated subunits. Such molecules include, but are not limited to, polypeptides, polynucleotides, polysaccharides or polyalkylene glycols. Polymers can also be biodegradable and/or biocompatible.

The terms “polypeptide,” “peptide,” and “protein” are used interchangeably herein and refer to a polymer of amino acid residues. The terms apply to naturally occurring amino acid polymers as well as amino acid polymers in which one or more amino acid residues are non-natural amino acids. Additionally, such polypeptides, peptides, and proteins include amino acid chains of any length, including full length proteins, wherein the amino acid residues are linked by covalent peptide bonds.

The term “drug,” as used herein, refers to any substance used in the prevention, diagnosis, alleviation, treatment, or cure of a disease or condition.

### Low-Aspect Ratio Nanostructures

The disclosure includes methods of designing and fabricating a new generation of hollow carbon nanostructures with unprecedented control in their length/diameter (L/D) aspect ratio, morphology, and dimension. Generally, the methods use well defined templates to make engineered structures of graphitic carbon with low aspect ratios.

One nonlimiting utility of the invention is to synthesize graphite nanocup, short carbon nanotube, and nanoring structures in a large scale while controlling their diameter (from 20 nm-100 nm), length (from 30 nm-1000 nm), and their combinations.

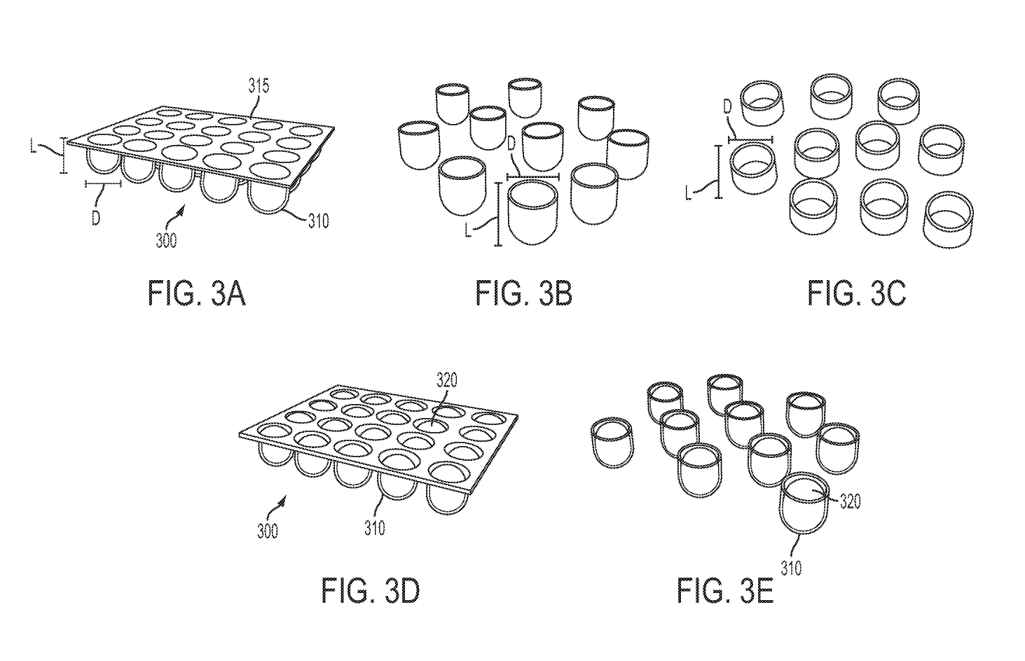

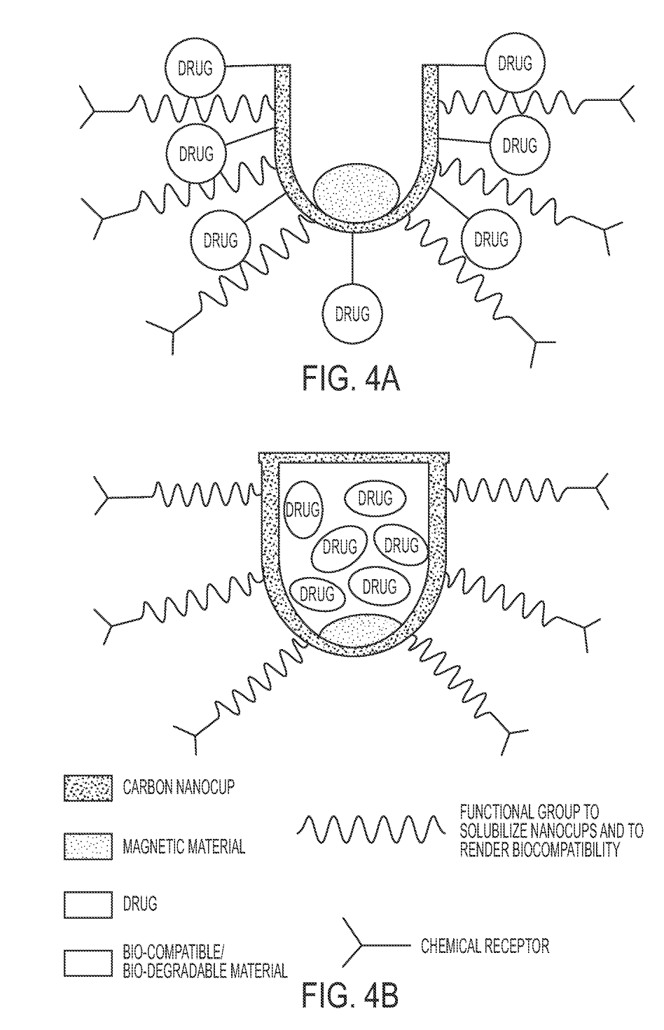

The disclosure includes the design and fabrication of a new generation of hollow carbon nanostructures with unprecedented control in their length/diameter (L/D) aspect ratio, morphology, and dimension. Morphologies that can be fabricated using the methods described herein include nanocups, nanorings, and continuous films of connected nanocups. Exemplary architectures include nanostructures fabricated from graphitic carbon, having up to 10⁵ times smaller length/diameter (L/D) ratios compared to conventional nanotubes, revealing unique morphologies of nanocups, nanorings, and large area connected nanocup arrays. Exemplary nanostructures are illustrated in FIG. 3.

As shown in FIG. 3A, array 300 can be fabricated having a plurality of nanocups 310 connected by layer 315 of carbon. Although array 300 is depicted having 20 nanocups 310, arrays can be fabricated having any number of nanocups. For example, an array can have about 2, about 5, about 10, about 20, about 30, about 40, about 50, about 60, about 70, about 80, about 90, about 100, about 200, about 400, about 500, about 1,000, or more nanocups. Other nonlimiting examples of arrays can have about 2 to about 1,000, about 100 to about 1,000, about 500 to about 10,000, about 1,000 to about 10,000, about 100 to about 500, about 500 to about 1,000, about 1,000 to about 5,000, or about 5,000 to about 10,000 nanocups.

Each nanocup 310 is hollow and has a length L and transverse diameter D. The length of the nanocup can be, e.g., about 20 nm, about 30 nm, about 40 nm, about 50 nm, about 60 nm, about 70 nm, about 80 nm, about 90 nm, about 100 nm, about 150 nm, about 200 nm, about 250 nm, about 300 nm, about 400 nm, about 500 nm, about 600 nm, about 700 nm, about 800 nm, about 900 nm, or about 1,000 nm. Other nonlimiting examples of nanocups have a length of about 20 nm to about 1,000 nm, about 40 nm to about 500 nm, about 60 nm to about 250 nm, about 80 nm to about 200 nm, about 20 nm to about 40 nm, about 50 nm to about 80 nm, about 100 nm to about 500 nm, or about 600 nm to about 1,000 nm. The transverse diameter can be, e.g., about 10 nm, about 20 nm, about 30 nm, about 40 nm, about 50 nm, about 60 nm, about 70 nm, about 80 nm, about 90 nm, about 100 nm, about 150 nm, about 200 nm, about 250 nm, about 300 nm, about 400 nm, or about 500 nm. Other nonlimiting examples of nanocups have a transverse diameter of about 10 nm to about 500 nm, about 20 nm to about 250 nm, about 40 nm to about 150 nm, about 10 nm to about 40 nm, about 50 nm to about 100 nm, about 110 nm to about 150 nm, about 160 nm to about 250 nm, or about 260 nm to about 500 nm.

Each nanocup has a low aspect ratio, defined as the ratio of length to transverse diameter. For example, the nanocup can have an aspect ratio of about 0.5, about 1, about 1.5, about 2, about 2.5, about 3, about 3.5, about 4, about 4.5, about 5, about 6, about 7, about 8, about 9, or about 10. Other nonlimiting examples of nanocups have an aspect ratio of about 0.5 to about 10, about 1 to about 5, about 1.5 to about 3, about 0.5 to about 1, about 1.5 to about 2, about 2 to about 3, about 3 to about 5, or about 6 to about 10.

Nanocups 310 can have walls of various thicknesses, which can depend on the amount of carbon deposited onto the underlying template. For example, the wall thickness can be about 1 nm, about 2 nm, about 3 nm, about 4 nm, about 5 nm, about 6 nm, about 7 nm, about 8 nm, about 9 nm, about 10 nm, about 15 nm, about 20 nm, about 25 nm, about 30 nm, about 35 nm, about 40 nm, about 50 nm, about 60 nm, about 70 nm, about 80 nm, about 90 nm, about 100 nm, about 150 nm, about 200 nm, or greater. Other nonlimiting examples of nanocups have a wall thickness of about 1 nm to about 200 nm, about 10 nm to about 100 nm, about 20 nm to about 50 nm, about 1 nm to about 5 nm, about 6 nm to about 10 nm, about 15 nm to about 50 nm, about 60 nm to about 100 nm, about 100 nm to about 150 nm, or about 150 nm to about 200 nm.

FIG. 3B illustrates exemplary separated nanocups, and FIG. 3C illustrates exemplary separated nanorings, each having a length L and a transverse diameter D. The separated nanocups and nanorings can have the same lengths, transverse diameters, aspect ratios, and wall thicknesses as described above for the nanocup arrays.

### Methods of Fabricating Low-Aspect Ratio Nanostructures

The methods described herein can be used to fabricate films (arrays) of connected nanostructures (such as connected nanocups) or separated nanostructures (such as separated nanocups and separated nanorings).

Generally, to fabricate arrays of connected nanostructures, the following protocol may be used: fabricating a template; using the template to fabricate nanostructure films; and removing the nanostructure film from the template.

Generally, to fabricate separated nanostructures, the method involves: fabricating a template; using the template to fabricate nanostructure films; separating individual nanostructures from the nanostructure film; and removing the individual nanostructures from the template.

Fabrication of Template

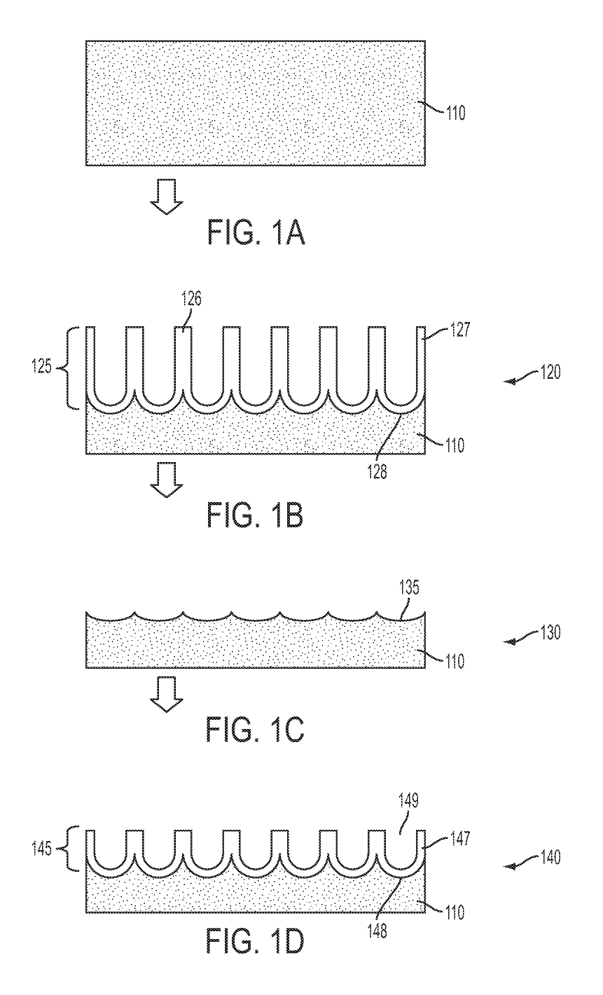

An exemplary method for fabricating a template is illustrated in FIG. 1. As depicted in FIG. 1A, aluminum sheet 110 is prepared. Aluminum sheet 110 can be a thin layer of aluminum, such as aluminum foil. Although the exemplary method utilizes aluminum, any metal that can be processed by electrochemical anodization can be used.

As illustrated in FIG. 1B, aluminum sheet 110 is subjected to electrochemical anodization, which results in structure 120. In one exemplary method, aluminum sheet 110 is subjected to anodization at 40-45V for 4 hours in 3% to 5% oxalic acid (C₂H₄O₂) solution at room temperature. Structure 120 includes an array of channels 125 within aluminum sheet 110. Channels 125 are defined by a layer 126 of aluminum oxide that forms lateral walls 127 and rounded bottoms 128 within aluminum sheet 110.

Next, as illustrated in FIG. 1C, layer 126 of aluminum oxide is removed from aluminum sheet 110. Layer 126 can be removed using known methods, such as, e.g., by contacting structure 120 with an acid solution (e.g., a solution of 5% phosphoric (H₃PO₄) and 5% chromic (H₂CrO₄) acid). This process results in structure 130, having a well-ordered array of grooves 135 within aluminum sheet 110, where at least a portion of lateral walls 127 and rounded bottoms 128 of channels 125 are negative templates for grooves 135.

As shown in FIG. 1D, structure 130 is then subjected to a second electrochemical anodization process. In one exemplary method, structure 130 is subjected to re-anodization for about 20 seconds to about 40 seconds. The re-anodization process results in final template 140, which contains nanochannels 145 within aluminum sheet 110. Nanochannels 145 are defined by lateral walls 147 and rounded bottoms 148, and are open at the top ends 149, opposite rounded bottoms 148. In exemplary methods, nanochannels 145 are about 20 nm, about 30 nm, about 40 nm, about 50 nm, about 60 nm, about 70 nm, about 80 nm, about 90 nm, about 100 nm, about 150 nm, about 200 nm, about 250 nm, about 300 nm, about 400 nm, about 500 nm, about 600 nm, about 700 nm, about 800 nm, about 900 nm, about 1,000 nm, about 10 nm to about 1,000 nm, about 20 nm to about 250 nm, about 40 nm to about 150 nm, about 10 nm to about 40 nm, about 50 nm to about 100 nm, about 110 nm to about 150 nm, about 160 nm to about 250 nm, or about 260 nm to about 500 nm long. In certain embodiments, the transverse diameters of nanochannels 145 are increased by acid treatment. For example, final template 140 can be treated with an acid, such as, but not limited to, sulfuric acid or phosphoric acid, to result increase the transverse diameter of nanochannels 145. In exemplary methods, the transverse diameter is about 10 nm, about 20 nm, about 30 nm, about 40 nm, about 50 nm, about 60 nm, about 70 nm, about 80 nm, about 90 nm, about 100 nm, about 150 nm, about 200 nm, about 250 nm, about 300 nm, about 400 nm, about 500 nm, about 10 nm to about 500 nm, about 20 nm to about 250 nm, about 40 nm to about 150 nm, about 10 nm to about 40 nm, about 50 nm to about 100 nm, about 110 nm to about 150 nm, about 160 nm to about 250 nm, or about 260 nm to about 500 nm.

Fabrication of Nanostructure Arrays Using Template

In certain instances, the methods described herein can be used to fabricate arrays of nanostructures, such as arrays of nanocups. Generally, an array of nanostructures comprises individual nanostructures connected together by a film, such as a thin graphite layer. In exemplary methods, the film flexibly tethers adjacent nanostructures to each other within the array.

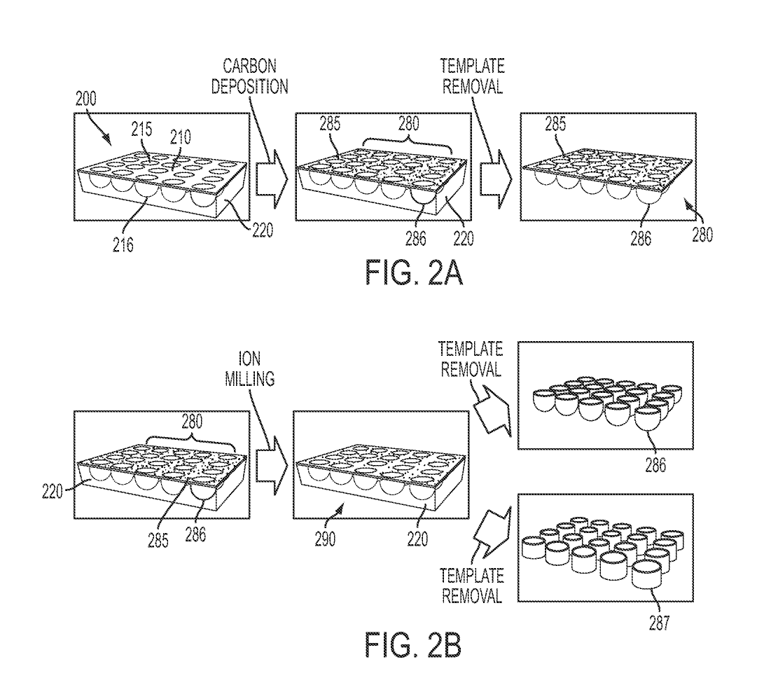

One exemplary method for fabricating arrays of nanostructures is illustrated in FIG. 2A. As depicted in FIG. 2A, template 200 is used to fabricate nanocup array 280. Template 200 is a template fabricated as described in FIG. 1. Template 200 includes a planar top surface 210, base 220, and nanochannels 215. Nanochannels 215 are open at top surface 210 and have rounded bottoms 216 enclosed within base 220. In a first step, carbon is deposited onto template 200. Upon deposition, the carbon contacts top surface 210 and the inner walls of nanochannels 215 of template 200, resulting in a thin layer of carbon deposited on template 200, as nanocup array 280, in association with template 200. The carbon can be deposited onto template 200 in a number of ways known in the art. One exemplary method is by chemical vapor deposition (CVD) (see, e.g., Kyotani et al. (1996) Chem. Mat. 8:2109-2113). Nanocup array 280 includes individual nanocups 286 connected to adjacent nanocups by a thin layer of carbon 285 disposed between adjacent nanocups 286 on the top surface 210 of template 200. Template 200 is then removed, resulting in the final nanocup array 280. In certain instances, template 200 is removed using a suitable acid solution, such as hydrofluoric acid.

Fabrication of Separated Nanostructures Using Template

In certain instances, the methods described herein can be used to fabricate separated or isolated nanostructures, such as nanocups or nanorings described herein. Generally, an array of nanostructures comprising individual nanostructures connected together by a film, such as a thin graphite layer, is fabricated, as described in FIG. 2. Prior to removal of the template from the array, the array is subjected to further processing, such as ion milling, resulting in the separation of nanostructures, such as nanocups and nanorings, from the array.

One exemplary method for fabricating separated nanocups and nanorings is illustrated in FIG. 2B. After forming a nanocup array 280 disposed on template 200 and having nanocups 286 connected by a thin layer of carbon 285 (as described in FIG. 2A), the nanocup array 280/template 200 assembly is subjected to ion milling, producing milled assembly 290. In certain embodiments, Ar ion milling can be used, although any inert gas can be used in the ion milling process. Other nonlimiting examples of inert gases include, e.g., N and He. The ion milling results in etching of the thin layer of carbon 285 between adjacent nanocups 286 in milled assembly 290, such that after removal of template 200, separated nanocups 286 are obtained.

The lengths of nanocups 286 are controlled by the duration of the ion milling process, and longer milling times can be used to fabricate shorter nanocups.

To produce separated nanorings 287, the nanocup array 280/template 200 assembly is subjected to ion milling for longer than the time for fabricating isolated nanocups. Such longer ion milling times result in the etching of the thin layer of carbon 285 between adjacent nanocups 286 in milled assembly 290, as well as etching of the rounded bottoms of nanocups 286 within base 220 of template 200. Removal of template 200 results in separated nanorings 287.

The ion milling times and conditions required to produce nanocups and nanorings of various sizes can be determined by the required dimensions of the final nanostructures. For example, about 70 seconds to about 90 seconds of ion milling can produce separated nanocups, and about 120 seconds to about 140 seconds of ion milling can produce separated nanorings.

### Low-Aspect Ratio Nanostructures for Agent Delivery

In certain embodiments, the nanocups are used to hold and contain agents, for example, metal nanoparticles, imaging agents, and/or therapeutic agents, leading to the formation of multi-component hybrid nanostructures.

FIG. 3D illustrates an exemplary nanoarray 300 of nanocups 310 filled with agent 320. FIG. 3E illustrates exemplary separated nanocups 310 filled with agent 320. In one embodiment, the agent is a metal, and the metal is deposited within the nanocup by electron beam evaporation. Other methods known in the art can also be used.

Such heterostructured constructs can be used, for example, to design pharmaceuticals for diagnostic purposes and can be developed as novel contrast agents for different imaging modalities. Also hybrid carbon nanocups that can be used in conjunction with magnetic resonance imaging (MRI) and inductive heating, such as in cancer treatment regimes.

The nanostructures described herein can also be functionalized and visualized (such as by inserting fluorescence nanoparticles) in biological environments using conventional fluorescence microscopy. As described herein, the nanostructures can be functionalized to target specific cells, become ingested, and then release their contents in response to a chemical trigger. Advantages of targeted drug delivery using the nanostructures described herein include the use of small quantities (such as nanogram levels) of drugs, reducing side effects. Further, the nanostructures described herein can isolate and contain an agent until reaching a target site, protecting the agent both from degradation and reaction with healthy cells.

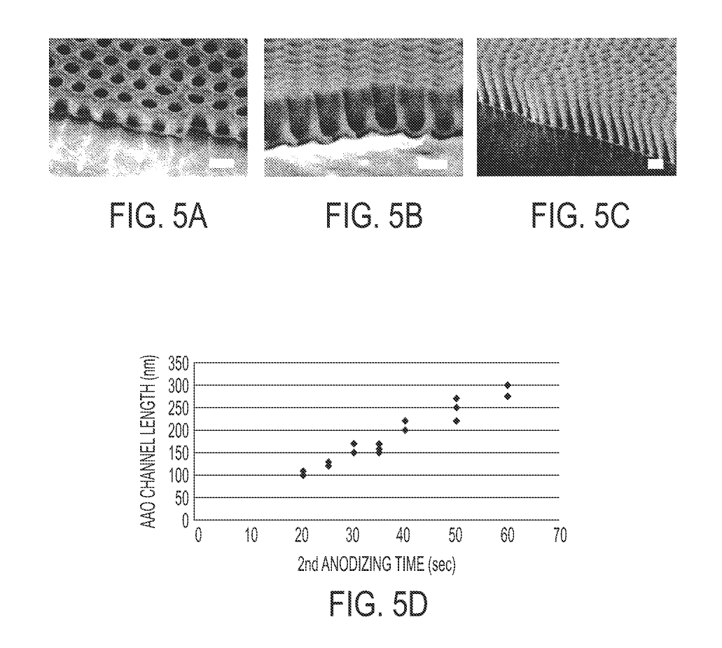

FIGS. 5A and 5B schematically illustrate exemplary nanocup based drug delivery systems, where a therapeutic agent is attached to the outer surface of a nanocup (FIG. 5A) or are inserted inside a carbon nanocup and capped with biodegradable material (FIG. 5B).

Other advantages of agent delivery using the nanostructures described herein include: the ability to control the inner volume in nanometer scale that can be filled with the desired chemical and functional nanoparticles; distinct inner and outer surfaces; an open end, which can make the inner volume and surfaces accessible; and a low aspect ratio, which can provide easy insertion of agents inside of a nanocup.

Fabrication of Carbon Nanocup Therapeutic Agent Delivery Vehicle

In particular embodiments, a carbon nanocup therapeutic agent delivery vehicle can be fabricated using one or more of the following steps. Carbon nanocups can be functionalized with a hydrophilic polymer such as poly ethylene glycol (PEG) for biocompatibility (soluble in water). The chemistry of carbon nanocups can allow the introduction of more than one function on the same cup (e.g., functionalizing the nanocup for both biocompatibility and targeting specific cells). Nanogram quantities of a therapeutic agent can be encapsulated within the nanocup interior by either simply filling with solution or by suction of the drug molecules, or drug molecules simply can be attached to outer wall of carbon nanocups. A functional group (e g , amino-terminated PEG) on the surface (e.g., introduced via a modified a-amino acid) can be used for conjugation of biological molecules such as peptide or drugs. An agent, such as doxorubicin, can be loaded on the surface of PEGylated nanocups. Once the agent is encapsulated or attached, the open end can be capped, which may be biodegradable or chemically removable. The nanocapsule can then be inserted into the body by intravenous injection or by oral administration. Due to the functionalized surface, the nanocapsule can target a designated site in the body. The cell can then internalize the nanocapsule by receptor-mediated endocytosis or by passive penetration. The cap then can be removed or can be biodegraded inside the cell, which can be caused by a chemical or external trigger (such as a change in pH, near-infrared radiation or chemically removable cap). Then, the drug molecules can be released into the cell by a cleavable bond or by a change in the local environment.

### Low-Aspect Ratio Nanostructures for Hydrogen Storage

Hydrogen has emerged as one of the most promising candidates for the replacement of the current carbon based energy source. Conventional hydrogen storages include compressed gas storage, cryogenic liquid hydrogen storage and metal hydride storage. Although each storage method provides desirable attributes, no approach satisfies the requirement of the efficiency, size, weight, cost and safety together. Recent advances in the science of carbon nanostructures have allowed new types of adsorbents to be engineered. Since carbon nanotubes have large surface area and abundant pore volume with relatively small mass, they have been considered as potential materials for high capacity hydrogen storage. There is a report that atomic hydrogen can be absorbed more preferentially at defect sites (dangling bonds) on carbon materials.

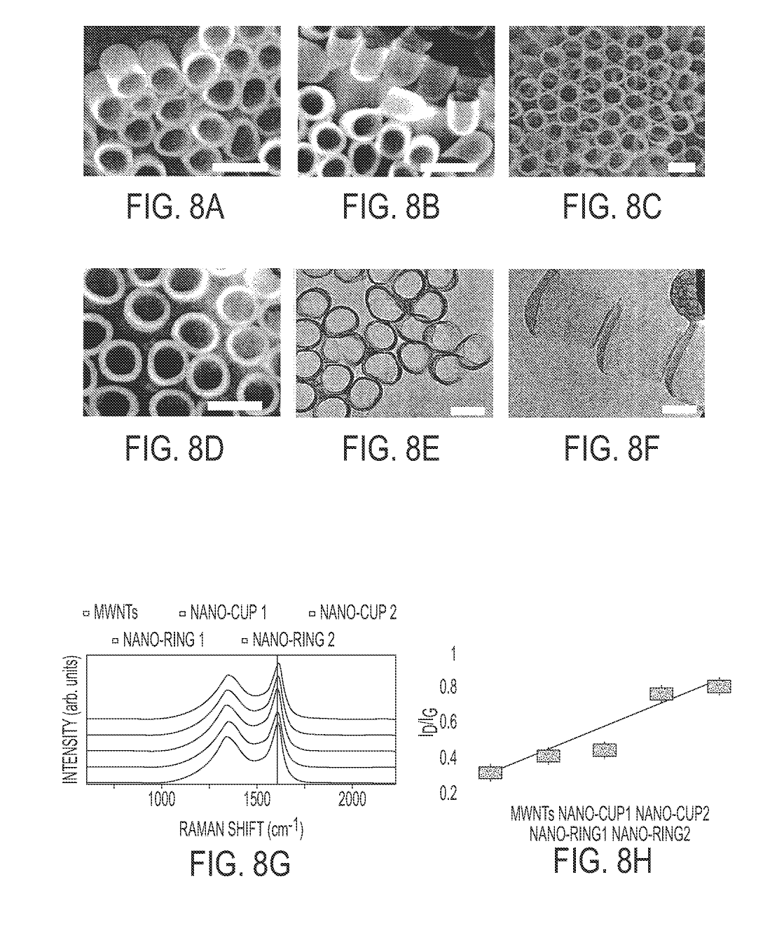

As shown in FIGS. 8G and 8H, Raman characterization of nanocups showed that the peak intensity ratio (ID/IG) doubled as the structure changed from long MWNTs to extremely short nanoring structures. This peak intensity ratio can be used as an index of CNTs quality, and it increases when disorder in a graphitic layer increases. This indicates that highly disordered lattice structures of nanocups were formed due to Ar ion irradiation and their extremely low length/diameter aspect ratio. Hence, these carbon nanocups can be used for hydrogen storage applications. Moreover, a wall thickness as well as morphology of carbon nanocups can be controlled to satisfy the target fundamental characteristics for the hydrogen storage application. In addition, nanocup-catalyst nanocluster hybrid structures can be fabricated that can effectively adsorb hydrogen atoms and molecules on carbon nanocup structures.

Nanocups can be subjected to hydrogenation, such as using a furnace connected with high purity H₂ and Ar gas cylinders. Nanocups can be heated at temperatures below about 873K under a mixture of H₂ and Ar atmosphere. These conditions can avoid recrystallization of the nanocups at high temperature as well as melting of Al substrates.

To tune H₂ storage capability of carbon nanocups, the morphology and structure of carbon nanocups can be controlled. Hydrogen atoms are trapped at graphite inter-layers and the edge surface of a crystallite through carbon dangling bonds. As discussed herein, the ion milling process as well as unique low aspect ratio of length/diameter of nanocups can increase the portion of dangling bonds on the edge of nanocup structures. To increase hydrogen storage capacity on nanocups, Ar ion-milling or oxygen plasma treatment can be applied for tailoring the edge and surface structure of carbon nanocups. The number of graphitic layers in nanocups can also enhance the hydrogen adsorption capacity on carbon nanocups. Thus, nanocups having a wall thickness (or arrays of such nanocups) as described herein can be used for hydrogen storage.

The content of nanostructured metal in carbon nanotubes can influence the hydrogen storage capacity. Metals such as Pd, Pt, Rh or Ru can act as a catalyst to assist the hydrogen attachment process. Pt dispersed in carbon fibers can dissociate hydrogen molecules into hydrogen atoms. Hydrogen atoms can subsequently spill onto nearby available storage sites on the nanocups, and can increase the hydrogen storage capacity of the nanocups. In certain embodiments, Pt nanoclusters of, e.g., about 1 nm to about 20 nm in size, can be formed on carbon nanocups using, e.g., ultra-high vacuum sputter deposition or electrochemical deposition. Other metals and other metal deposition methods known in the art can also be used.

### Therapeutic and Detection Agents

A nanostructure fabricated using a method described herein can be modified with many types of compounds, such as, but not limited to, therapeutic or detection agents. A nanostructure can be modified by inserting an agent into the hollow space within the nanostructure (such as within a nanocup), and/or a nanostructure can be modified by attaching an agent to a surface of a nanostructure, such as an outer or an inner surface.

Nonlimiting examples of therapeutic agents include, e.g., steroids, analgesics, local anesthetics, antibiotic agents, chemotherapeutic agents, immunosuppressive agents, anti-inflammatory agents, antiproliferative agents, antimitotic agents, angiogenic agents, antipsychotic agents, central nervous system (CNS) agents, anticoagulants, fibrinolytic agents, growth factors, antibodies, ocular drugs, and metabolites, analogs, derivatives, fragments, and purified, isolated, recombinant and chemically synthesized versions of these species, and combinations thereof.

Representative useful therapeutic agents include, but are not limited to, tamoxifen, paclitaxel, low soluble anticancer drugs, camptothecin and its derivatives, e.g., topotecan and irinotecan, KRN 5500 (KRN), meso-tetraphenylporphine, dexamethasone, benzodiazepines, allopurinol, acetohexamide, benzthiazide, chlorpromazine, chlordiazepoxide, haloperidol, indomethacine, lorazepam, methoxsalen, methylprednisone, nifedipine, oxazepam, oxyphenbutazone, prednisone, prednisolone, pyrimethamine, phenindione, sulfisoxazole, sulfadiazine, temazepam, sulfamerazine, ellipticin, porphine derivatives for photo-dynamic therapy, and/or trioxsalen, as well as all mainstream antibiotics, including the penicillin group, fluoroquinolones, and first, second, third, and fourth generation cephalosporins. These agents are commercially available from, e.g., Merck & Co., Barr Laboratories, Avalon Pharma, and Sun Pharma, among others.

In some instances, the nanostructures described herein can be used to detect or image cells, e.g., using nanostructures with a detection agent inserted into the hollow space or using nanostructures coupled to a detection agent (e.g., on an outer surface or inner surface). The detection agent can be used to qualitatively or quantitatively analyze the location and/or the amount of a nanostructure at a particular locus. The detection agent can also be used to image a nanostructure and/or a cell or tissue target of a nanostructure using standard methods.

In some instances, the nanostructures are derivatized (or labeled) with a detection agent. Examples of detection agents include magnetic resonance imaging contrast agents, computed tomography (CT scan) imaging agents, optical imaging agents and radioisotopes. Nonlimiting examples of detection agents include, without limitation, fluorescent compounds, various enzymes, prosthetic groups, luminescent materials, bioluminescent materials, fluorescent emitting metal atoms, (e.g., europium (Eu)), radioactive isotopes (described below), quantum dots, electron-dense reagents, and haptens. The detection reagent can be detected using various means including, but are not limited to, spectroscopic, photochemical, radiochemical, biochemical, immunochemical, or chemical means.

Nonlimiting exemplary fluorescent detection agents include fluorescein, fluorescein isothiocyanate, rhodamine, 5-dimethylamine-1-napthalenesulfonyl chloride, phycoerythrin, and the like. A detection agent can also be a detectable enzyme, such as alkaline phosphatase, horseradish peroxidase, β-galactosidase, acetylcholinesterase, glucose oxidase and the like. When a nanostructure is derivatized with a detectable enzyme, it can be detected by adding additional reagents that the enzyme uses to produce a detectable reaction product. For example, when the detection agent is horseradish peroxidase, the addition of hydrogen peroxide and diaminobenzidine leads to a detectable colored reaction product. A nanostructure can also be derivatized with a prosthetic group (e.g., streptavidin/biotin and avidin/biotin). For example, a nanostructure can be derivatized with biotin and detected through indirect measurement of avidin or streptavidin binding. Nonlimiting examples of fluorescent compounds that can be used as detection reagents include umbelliferone, fluorescein, fluorescein isothiocyanate, rhodamine, dichlorotriazinylamine fluorescein, dansyl chloride, and phycoerythrin. Luminescent materials include, e g., luminol, and bioluminescent materials include, e.g., luciferase, luciferin, and aequorin.

A detection agent can also be a radioactive isotope, such as, but not limited to, α-, β-, or γ-emitters; or β- and γ-emitters. Radioactive isotopes can be used in diagnostic or therapeutic applications. Such radioactive isotopes include, but are not limited to, iodine (¹³¹I or ¹²⁵I), yttrium (⁹⁰Y), lutetium (¹⁷⁷Lu), actinium (²²⁵Ac), praseodymium (¹⁴²Pr or ¹⁴³Pr), astatine (²¹¹At,) rhenium (¹⁸⁶Re or ¹⁸⁷Re), bismuth (²¹²Bi or ²¹³Bi), indium (¹¹¹In), technetium (^{99m}Tc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), and gallium (⁶⁷Ga).

The nanostructure can be radiolabeled using techniques known in the art. In some situations, a nanostructure described herein is contacted with a chelating agent, e.g., 1,4,7,10-tetraazacyclododecane-N,N′,N″,N′″-tetraacetic acid (DOTA), to thereby produce a conjugated nanostructure. The conjugated nanostructure is then radiolabeled with a radioisotope, e.g., ¹¹¹In, ⁹⁰Y, ¹⁷⁷Lu, ¹⁸⁶Re, ¹⁸⁷Re, or ^{99m}Tc, to thereby produce a labeled nanostructure. In other methods, the nanostructures can be labeled with ¹¹¹In and ⁹⁰Y using weak transchelators such as citrate (see, e.g., Khaw et al., Science 209:295-297 (1980)) or ^{99m}Tc after reduction in reducing agents such as Na Dithionite (see, e.g., Khaw et al., J. Nucl. Med. 23:1011-1019 (1982)) or by SnCl₂ reduction (see, e.g., Khaw et al., J. Nucl. Med. 47:868-876 (2006)). Other methods are described in, e.g., Lindegren et al., Bioconjug. Chem. 13:502-509 (2002); Boyd et al., Mol. Pharm. 3:614-627 (2006); and del Rosario et al., J. Nucl. Med. 34:1147-1151 (1993).

### Polymers

In certain instances, biodegradable and/or biocompatible polymers are used, such as for capping and sealing a nanocup described herein. These include, without limitation, substantially pure carbon lattices (e.g., graphite), dextran, polysaccharides, polypeptides, polynucleotides, acrylate gels, polyanhydride, poly(lactide-co-glycolide), polytetraflouroethylene, polyhydroxyalkonates, cross-linked alginates, gelatin, collagen, cross-linked collagen, collagen derivatives (such as succinylated collagen or methylated collagen), cross-linked hyaluronic acid, chitosan, chitosan derivatives (such as methylpyrrolidone-chitosan), cellulose and cellulose derivatives (such as cellulose acetate or carboxymethyl cellulose), dextran derivatives (such carboxymethyl dextran), starch and derivatives of starch (such as hydroxyethyl starch), other glycosaminoglycans and their derivatives, other polyanionic polysaccharides or their derivatives, polylactic acid (PLA), polyglycolic acid (PGA), a copolymer of a polylactic acid and a polyglycolic acid (PLGA), lactides, glycolides, and other polyesters, polyglycolide homoploymers, polyoxanones and polyoxalates, copolymer of poly(bis(p-carboxyphenoxy)propane)anhydride (PCPP) and sebacic acid, poly(l-glutamic acid), poly(d-glutamic acid), polyacrylic acid, poly(dl-glutamic acid), poly(l-aspartic acid), poly(d-aspartic acid), poly(dl-aspartic acid), polyethylene glycol, copolymers of the above listed polyamino acids with polyethylene glycol, polypeptides, such as, collagen-like, silk-like, and silk-elastin-like proteins, polycaprolactone, poly(alkylene succinates), poly(hydroxy butyrate) (PHB), poly(butylene diglycolate), nylon-2/nylon-6-copolyamides, polydihydropyrans, polyphosphazenes, poly(ortho ester), poly(cyano acrylates), polyvinylpyrrolidone, polyvinylalcohol, poly casein, keratin, myosin, and fibrin, silicone rubbers, or polyurethanes, and the like. Other biodegradable materials that can be used include naturally derived polymers, such as acacia, gelatin, dextrans, albumins, alginates/starch, and the like; or synthetic polymers, whether hydrophilic or hydrophobic. The materials can be synthesized, isolated, and are commercially available.

### Targeting Agents

In some instances, a nanostructure described herein includes a targeting agent that is attached, fixed, or conjugated to, the outermost surface of the nanostructure. In certain situations, the targeting agent specifically binds to a particular biological target. Nonlimiting examples of biological targets include tumor cells, bacteria, viruses, cell surface proteins, cell surface receptors, cell surface polysaccharides, extracellular matrix proteins, intracellular proteins and intracellular nucleic acids. The targeting agents can be, for example, various specific ligands, such as antibodies, monoclonal antibodies and their fragments, folate, mannose, galactose and other mono-, di-, and oligosaccharides, and RGD peptide.

The nanostructures and methods described herein are not limited to any particular targeting agent, and a variety of targeting agents can be used. Examples of such targeting agents include, but are not limited to, nucleic acids (e.g., RNA and DNA), polypeptides (e.g., receptor ligands, signal peptides, avidin, Protein A, and antigen binding proteins), polysaccharides, biotin, hydrophobic groups, hydrophilic groups, drugs, and any organic molecules that bind to receptors. In some instances, a nanostructure described herein can be conjugated to one, two, or more of a variety of targeting agents. For example, when two or more targeting agents are used, the targeting agents can be similar or dissimilar. Utilization of more than one targeting agent on a particular nanostructure can allow the targeting of multiple biological targets or can increase the affinity for a particular target.

The targeting agents can be associated with the nanostructures in a number of ways. For example, the targeting agents can be associated (e.g., covalently or noncovalently bound) to other subcomponents/elements of the nanostructure with either short (e.g., direct coupling), medium (e.g., using small-molecule bifunctional linkers such as SPDP (Pierce Biotechnology, Inc., Rockford, Ill.)), or long (e.g., PEG bifunctional linkers (Nektar Therapeutics, Inc., San Carlos, Calif.)) linkages. Alternatively, such agents can be directly conjugated to the outer surface of a nanostructure.

In addition, a nanostructure can also incorporate reactive groups (e.g., amine groups such as polylysine, dextranemine, profamine sulfate, and/or chitosan). The reactive group can allow for further attachment of various specific ligands or reporter groups (e.g., ¹²⁵I, ¹³¹I, Br, various chelating groups such as DTPA, which can be loaded with reporter heavy metals such as ¹¹¹In, ^{99m}Tc, GD, Mn, fluorescent groups such as FITC, rhodamine, Alexa, and quantum dots), and/or other moieties (e.g., ligands, antibodies, and/or portions thereof).

Antibodies as Targeting Agents

In some instances, the targeting agents are antigen binding proteins or antibodies or binding portions thereof Antibodies can be generated to allow for the specific targeting of antigens or immunogens (e.g., tumor, tissue, or pathogen specific antigens) on various biological targets (e.g., pathogens, tumor cells, normal tissue). Such antibodies include, but are not limited to, polyclonal antibodies; monoclonal antibodies or antigen binding fragments thereof; modified antibodies such as chimeric antibodies, reshaped antibodies, humanized antibodies, or fragments thereof (e.g., Fv, Fab', Fab, F(ab')₂); or biosynthetic antibodies, e.g., single chain antibodies, single domain antibodies (DAB), Fvs, or single chain Fvs (scFv).

Methods of making and using polyclonal and monoclonal antibodies are well known in the art, e.g., in Harlow et al., Using Antibodies: A Laboratory Manual: Portable Protocol I. Cold Spring Harbor Laboratory (Dec. 1, 1998). Methods for making modified antibodies and antibody fragments (e.g., chimeric antibodies, reshaped antibodies, humanized antibodies, or fragments thereof, e.g., Fab', Fab, F(ab')₂ fragments); or biosynthetic antibodies (e.g., single chain antibodies, single domain antibodies (DABs), Fv, single chain Fv (scFv), and the like), are known in the art and can be found, e.g., in Zola, Monoclonal Antibodies: Preparation and Use of Monoclonal Antibodies and Engineered Antibody Derivatives, Springer Verlag (Dec. 15, 2000; 1st edition).

Antibody attachment to nanostructures can be performed through standard covalent binding to free amine groups (see, e.g., Torchilin et al. (1987) Hybridoma, 6:229-240; Torchilin, et al., (2001) Biochim. Biophys. Acta, 1511:397-411; Masuko, et al., (2005), Biomacromol., 6:800-884) in the outermost surface of the nanostructure. Standard methods of protein covalent binding are known, such as covalent binding through amine groups. This methodology can be found in, e.g., Protein Architecture: Interfacing Molecular Assemblies and Immobilization, editors: Lvov et al. (2000) Chapter 2, pp. 25-54. In certain instances, the outer surface of the nanostructure can be functionalized with a polymer that has free amino, carboxy, SH-, epoxy-, and/or other groups that can react with ligand molecules directly or after preliminary activation with, e.g., carbodiimides, SPDP, SMCC, and/or other mono- and bifunctional reagents.

Signal Peptides as Targeting Agents

In some instances, the targeting agents include a signal peptide. These peptides can be chemically synthesized or cloned, expressed and purified using known techniques. Signal peptides can be used to target the nanoparticles described herein to a discreet region within a cell. In some situations, specific amino acid sequences are responsible for targeting the nanoparticles into cellular organelles and compartments. For example, the signal peptides can direct a nanoparticle described herein into mitochondria. In other examples, a nuclear localization signal is used.

Nucleic Acids as Targeting Agents

In other instances, the targeting agent is a nucleic acid (e.g., RNA or DNA). In some examples, the nucleic acid targeting agents are designed to hybridize by base pairing to a particular nucleic acid (e.g., chromosomal DNA, mRNA, or ribosomal RNA). In other situations, the nucleic acids bind a ligand or biological target. For example, the nucleic acid can bind reverse transcriptase, Rev or Tat proteins of HIV (Tuerk et al., Gene, 137(1):33-9 (1993)); human nerve growth factor (Binkley et al., Nuc. Acids Res., 23(16):3198-205 (1995)); or vascular endothelial growth factor (Jellinek et al., Biochem., 83(34): 10450-6 (1994)). Nucleic acids that bind ligands can be identified by known methods, such as the SELEX procedure (see, e.g., U.S. Pat. Nos. 5,475,096; 5,270,163; and 5,475,096; and WO 97/38134; WO 98/33941; and WO 99/07724). The targeting agents can also be aptamers that bind to particular sequences.

Other Targeting Agents

The targeting agents can recognize a variety of epitopes on preselected biological targets (e.g., pathogens, tumor cells, or normal cells). For example, in some instances, the targeting agent can be sialic acid to target HIV (Wies et al., Nature, 333:426 (1988)), influenza (White et al., Cell, 56:725 (1989)), Chlamydia (Infect. Immunol, 57:2378 (1989)), Neisseria meningitidis, Streptococcus suis, Salmonella, mumps, newcastle, reovirus, Sendai virus, and myxovirus; and 9-OAC sialic acid to target coronavirus, encephalomyelitis virus, and rotavirus; non-sialic acid glycoproteins to target cytomegalovirus (Virology, 176:337 (1990)) and measles virus (Virology, 172:386 (1989)); CD4 (Khatzman et al., Nature, 312:763 (1985)), vasoactive intestinal peptide (Sacerdote et al., J. of Neuroscience Research, 18:102 (1987)), and peptide T (Ruff et al., FEBS Letters, 211:17 (1987)) to target HIV; epidermal growth factor to target vaccinia (Epstein et al., Nature, 318: 663 (1985)); acetylcholine receptor to target rabies (Lentz et al., Science 215: 182 (1982)); Cd3 complement receptor to target Epstein-Barr virus (Carel et al., J. Biol. Chem., 265:12293 (1990)); .beta.-adrenergic receptor to target reovirus (Co et al., Proc. Natl. Acad. Sci. USA, 82:1494 (1985)); ICAM-1 (Marlin et al., Nature, 344:70 (1990)), N-CAM, and myelin-associated glycoprotein MAb (Shephey et al., Proc. Natl. Acad. Sci. USA, 85:7743 (1988)) to target rhinovirus; polio virus receptor to target polio virus (Mendelsohn et al., Cell, 56:855 (1989)); fibroblast growth factor receptor to target herpes virus (Kaner et al., Science, 248:1410 (1990)); oligomannose to target Escherichia coli; and ganglioside G_{M1} to target Neisseria meningitides.

In other instances, the targeting agent targets nanostructures to factors expressed by oncogenes. These can include, but are not limited to, tyrosine kinases (membrane-associated and cytoplasmic forms), such as members of the Src family; serine/threonine kinases, such as Mos; growth factor and receptors, such as platelet derived growth factor (PDDG), SMALL GTPases (G proteins), including the ras family, cyclin-dependent protein kinases (cdk), members of the myc family members, including c-myc, N-myc, and L-myc, and bcl-2 family members.

In addition, vitamins (both fat soluble and non-fat soluble vitamins) can be used as targeting agents to target biological targets (e.g., cells) that have receptors for, or otherwise take up, vitamins. For example, fat soluble vitamins (such as vitamin D and its analogs, vitamin E, vitamin A), and water soluble vitamins (such as vitamin C) can be used as targeting agents.

### Therapeutic Administration

The nanostructures described herein can be used to treat (e.g., mediate the translocation of drugs into) diseased cells and tissues. In this regard, various diseases are amenable to treatment using the nanostructures and methods described herein. Exemplary, nonlimiting diseases that can be treated with the nanostructures include breast cancer; prostate cancer; lung cancer; lymphomas; skin cancer; pancreatic cancer; colon cancer; melanoma; ovarian cancer; brain cancer; head and neck cancer; liver cancer; bladder cancer; non-small lung cancer; cervical carcinoma; leukemia; non-Hodgkins lymphoma, multiple sclerosis, neuroblastoma and glioblastoma; T and B cell mediated autoimmune diseases; inflammatory diseases; infections; hyperproliferative diseases; AIDS; degenerative conditions, cardiovascular diseases, transplant rejection, and the like. In some cases, the treated cancer cells are metastatic.

The route and/or mode of administration of a nanostructure described herein can vary depending upon the desired results. Dosage regimens can be adjusted to provide the desired response, e.g., a therapeutic response.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. The mode of administration is left to the discretion of the practitioner.

In some instances, a nanostructure described herein is administered locally. This is achieved, for example, by local infusion during surgery, topical application (e.g., in a cream or lotion), by injection, by means of a catheter, by means of a suppository or enema, or by means of an implant, said implant being of a porous, non-porous, or gelatinous material, including membranes, such as sialastic membranes, or fibers. In some situations, a nanostructure described herein is introduced into the central nervous system, circulatory system or gastrointestinal tract by any suitable route, including intraventricular, intrathecal injection, paraspinal injection, epidural injection, enema, and by injection adjacent to the peripheral nerve. Intraventricular injection can be facilitated by an intraventricular catheter, for example, attached to a reservoir, such as an Ommaya reservoir.

This disclosure also features a device for administering a nanostructure described herein. The device can include, e.g., one or more housings for storing pharmaceutical compositions, and can be configured to deliver unit doses of a nanostructure described herein.

Pulmonary administration can also be employed, e.g., by use of an inhaler or nebulizer, and formulation with an aerosolizing agent, or via perfusion in a fluorocarbon or synthetic pulmonary surfactant.

In some instances, a nanostructure described herein can be delivered in a vesicle, in particular, a liposome (see Langer, Science 249:1527-1533 (1990) and Treat et al., Liposomes in the Therapy of Infectious Disease and Cancer pp. 317-327 and pp. 353-365 (1989)).

In yet other situations, a nanostructure described herein can be delivered in a controlled-release system or sustained-release system (see, e.g., Goodson, in Medical Applications of Controlled Release, vol. 2, pp. 115-138 (1984)). Other controlled or sustained-release systems discussed in the review by Langer, Science 249:1527-1533 (1990) can be used. In one case, a pump can be used (Langer, Science 249:1527-1533 (1990); Sefton, CRC Crit. Ref Biomed. Eng. 14:201 (1987); Buchwald et al., Surgery 88:507 (1980); and Saudek et al., N. Engl. J. Med. 321:574 (1989)).

In yet other situations, a controlled- or sustained-release system can be placed in proximity of a target of nanostructure described herein, reducing the dose to a fraction of the systemic dose.

A nanostructure described herein can be formulated as a pharmaceutical composition that includes a suitable amount of a physiologically acceptable excipient (see, e.g., Remington's Pharmaceutical Sciences pp. 1447-1676 (Alfonso R. Gennaro, ed., 19th ed. 1995)). Such physiologically acceptable excipients can be, e.g., liquids, such as water and oils, including those of petroleum, animal, vegetable, or synthetic origin, such as peanut oil, soybean oil, mineral oil, sesame oil and the like. The physiologically acceptable excipients can be saline, gum acacia, gelatin, starch paste, talc, keratin, colloidal silica, urea and the like. In addition, auxiliary, stabilizing, thickening, lubricating, and coloring agents can be used. In one situation, the physiologically acceptable excipients are sterile when administered to an animal. The physiologically acceptable excipient should be stable under the conditions of manufacture and storage and should be preserved against the contaminating action of microorganisms. Water is a particularly useful excipient when a nanostructure described herein is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Other examples of suitable physiologically acceptable excipients are described in Remington's Pharmaceutical Sciences pp. 1447-1676 (Alfonso R. Gennaro, ed., 19th ed. 1995). The pharmaceutical compositions, if desired, can also contain minor amounts of wetting or emulsifying agents, or pH buffering agents.

Liquid carriers can be used in preparing solutions, suspensions, emulsions, syrups, and elixirs. A nanostructure described herein can be suspended in a pharmaceutically acceptable liquid carrier such as water, an organic solvent, a mixture of both, or pharmaceutically acceptable oils or fat. The liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particular containing additives described herein, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. The liquid carriers can be in sterile liquid form for administration. The liquid carrier for pressurized compositions can be halogenated hydrocarbon or other pharmaceutically acceptable propellant.

In other instances, a nanostructure described herein is formulated for intravenous administration. Compositions for intravenous administration can comprise a sterile isotonic aqueous buffer. The compositions can also include a solubilizing agent. Compositions for intravenous administration can optionally include a local anesthetic such as lignocaine to lessen pain at the site of the injection. The ingredients can be supplied either separately or mixed together in unit dosage form, for example, as a dry lyophilized powder or water-free concentrate in a hermetically sealed container such as an ampule or sachette indicating the quantity of active agent. Where a nanostructure described herein is administered by infusion, it can be dispensed, for example, with an infusion bottle containing sterile pharmaceutical grade water or saline. Where a nanostructure described herein is administered by injection, an ampule of sterile water for injection or saline can be provided so that the ingredients can be mixed prior to administration.

In other circumstances, a nanostructure described herein can be administered across the surface of the body and the inner linings of the bodily passages, including epithelial and mucosal tissues. Such administrations can be carried out using a nanostructure described herein in lotions, creams, foams, patches, suspensions, solutions, and suppositories (e.g., rectal or vaginal). In some instances, a transdermal patch can be used that contains a nanostructure described herein and a carrier that is inert to the nanostructure described herein, is non-toxic to the skin, and that allows delivery of the agent for systemic absorption into the blood stream via the skin. The carrier can take any number of forms such as creams or ointments, pastes, gels, or occlusive devices. The creams or ointments can be viscous liquid or semisolid emulsions of either the oil-in-water or water-in-oil type. Pastes of absorptive powders dispersed in petroleum or hydrophilic petroleum containing a nanostructure described herein can also be used. A variety of occlusive devices can be used to release a nanostructure described herein into the blood stream, such as a semi-permeable membrane covering a reservoir containing the nanostructure with or without a carrier, or a matrix containing the nanostructure.

A nanostructure described herein can be administered rectally or vaginally in the form of a conventional suppository. Suppository formulations can be made using methods known to those in the art from traditional materials, including cocoa butter, with or without the addition of waxes to alter the suppository's melting point, and glycerin. Water-soluble suppository bases, such as polyethylene glycols of various molecular weights, can also be used.

The amount of a nanostructure described herein that is effective for treating disorder or disease can be determined using standard clinical techniques known to those with skill in the art. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. For example, the dose of a nanostructure described herein can each range from about 0.001 mg/kg to about 250 mg/kg of body weight per day, from about 1 mg/kg to about 250 mg/kg body weight per day, from about 1 mg/kg to about 50 mg/kg body weight per day, or from about 1 mg/kg to about 20 mg/kg of body weight per day. Equivalent dosages can be administered over various time periods including, but not limited to, about every 2 hrs, about every 6 hrs, about every 8 hrs, about every 12 hrs, about every 24 hrs, about every 36 hrs, about every 48 hrs, about every 72 hrs, about every week, about every two weeks, about every three weeks, about every month, and about every two months. The number and frequency of dosages corresponding to a completed course of therapy can be determined according to the judgment of a health-care practitioner.

In some instances, a pharmaceutical composition described herein is in unit dosage form, e.g., as a tablet, capsule, powder, solution, suspension, emulsion, granule, or suppository. In such form, the pharmaceutical composition can be sub-divided into unit doses containing appropriate quantities of a nanoparticle described herein. The unit dosage form can be a packaged pharmaceutical composition, for example, packeted powders, vials, ampoules, pre-filled syringes or sachets containing liquids. The unit dosage form can be, for example, a capsule or tablet itself, or it can be the appropriate number of any such compositions in package form. Such unit dosage form can contain from about 1 mg/kg to about 250 mg/kg, and can be given in a single dose or in two or more divided doses.

### Kits

A nanostructure described herein can be provided in a kit. In some instances, the kit includes (a) a container that contains a nanostructure and, optionally (b) informational material. The informational material can be descriptive, instructional, marketing or other material that relates to the methods described herein and/or the use of the nanostructures, e.g., for therapeutic benefit.

The informational material of the kits is not limited in its form. In some instances, the informational material can include information about production of the nanostructure, molecular weight of the nanostructure, concentration, date of expiration, batch or production site information, and so forth. In other situations, the informational material relates to methods of administering the nanostructures, e.g., in a suitable amount, manner, or mode of administration (e.g., a dose, dosage form, or mode of administration described herein). The method can be a method of treating a subject having a disorder.

In some cases, the informational material, e.g., instructions, is provided in printed matter, e.g., a printed text, drawing, and/or photograph, e.g., a label or printed sheet. The informational material can also be provided in other formats, such as Braille, computer readable material, video recording, or audio recording. In other instances, the informational material of the kit is contact information, e.g., a physical address, email address, website, or telephone number, where a user of the kit can obtain substantive information about the nanostructures therein and/or their use in the methods described herein. The informational material can also be provided in any combination of formats.

In addition to the nanostructures, the kit can include other ingredients, such as a solvent or buffer, a stabilizer, or a preservative. The kit can also include other agents, e.g., a second or third agent, e.g., other therapeutic agents. The components can be provided in any form, e.g., liquid, dried or lyophilized form. The components can be substantially pure (although they can be combined together or delivered separate from one another) and/or sterile. When the components are provided in a liquid solution, the liquid solution can be an aqueous solution, such as a sterile aqueous solution. When the components are provided as a dried form, reconstitution generally is by the addition of a suitable solvent. The solvent, e.g., sterile water or buffer, can optionally be provided in the kit.

The kit can include one or more containers for the nanostructures or other agents. In some cases, the kit contains separate containers, dividers or compartments for the nanostructures and informational material. For example, the nanostructures can be contained in a bottle, vial, or syringe, and the informational material can be contained in a plastic sleeve or packet. In other situations, the separate elements of the kit are contained within a single, undivided container. For example, the nanostructures can be contained in a bottle, vial or syringe that has attached thereto the informational material in the form of a label. In some cases, the kit can include a plurality (e.g., a pack) of individual containers, each containing one or more unit dosage forms (e.g., a dosage form described herein) of the nanostructures. The containers can include a unit dosage, e.g., a unit that includes the nanostructures. For example, the kit can include a plurality of syringes, ampules, foil packets, blister packs, or medical devices, e.g., each containing a unit dose. The containers of the kits can be air tight, waterproof (e.g., impermeable to changes in moisture or evaporation), and/or light-tight.

The kit can optionally include a device suitable for administration of the nanostructures, e.g., a syringe or other suitable delivery device. The device can be provided pre-loaded with nanostructures, e.g., in a unit dose, or can be empty, but suitable for loading.

The invention is further illustrated by the following examples. The examples are provided for illustrative purposes only. They are not to be construed as limiting the scope or content of the invention in any way.

### EXAMPLES

### EXAMPLE 1

### Preparation of Low-Aspect Ratio Carbon Nanostructures

### A. Methods

Fabrication of Anodized Aluminum Oxide (AAO) Template

In order to produce highly ordered arrays of nanopores, a two-step anodization process was used. In the first step, a high purity Al foil (Alfa Aesar, 99.99%) was anodized at 40-45V for 4 hours in 3-5% oxalic acid (C₂H₄O₂) solution at room temperature (RT). The anodized Al film was placed in the solution containing a mixture of 5% phosphoric (H₃PO₄) and 5% chromic (H₂CrO₄) acids for 24 hours to remove the formed aluminum oxide layer. This process resulted in the formation of a well-ordered array of scallop-shapes on the aluminum surface.

A re-anodization process was then performed but in precisely controlled and short time (for 20 sec to 40 sec) to fabricate highly organized short nanochannels (80 nm-200 nm in length) giving 10³-10⁵ time smaller L/D aspect ratio. Then, samples were soaked in a 5% phosphoric acid solution for 1 hour, which resulted in the widening of nanopores.

Fabrication of Carbon Nanostructures Using AAO Template

Low aspect-ratio carbon nanostructures were synthesized by using a chemical vapor deposition (CVD) process (Kyotani et al. (1996) Chem. Mat. 8:2109-2113). The AAO template was first placed in a quartz tube, and evacuated to 15 mTorr. During heat-up, high purity argon gas (99.9%) was supplied and the pressure was maintained at 760 Torr. When the temperature of the inside quartz tube reached 660° C., acetylene (5 sccm)-argon (45 sccm) mixture gas was supplied as a carbon source for the deposition of a graphitic carbon layer inside the low-aspect ratio nanochannels within the AAO template, resulting in the connected arrays of carbon nanocup film structures.

Fabrication of Separated Carbon Nanocups and Nanorings

In order to fabricate separated and length controlled nanocup and nanoring structures, Ar ion-milling was used. The connected carbon nanocup film was loaded inside of an ion milling chamber with a 90° incident angle, and the chamber was evacuated to 5×10⁻⁶ Torr. Next, 35 sccm of argon was flowed into the system, creating a 2×10⁻⁴ Torr working pressure. The 250 V beam voltage and the 55 mA beam current pushed electrons off the filament to ionize the argon atoms. The accelerating voltage was set to 300 V to accelerate the argon cations. Ar ion-milling process was run for 70 sec to 90 sec, and 120 sec to 140 sec to fabricate and control the lengths of separated nanocups and nanorings, respectively.

Fabrication of Metal Nanoparticle-Nanocup Heterostructures

Gold with 80 nm thickness was deposited on the carbon nanocup structures (both connected arrays and individually separated ones) inside of an AAO template using electron beam evaporation. The gold-deposited carbon nanocup structures were annealed at 600° C. for 6 hours under Ar atmospheric environment. For lead, 60 nm thick films were deposited on the carbon nanocup structures inside of an AAO template using a thermal evaporator, and then it was annealed at 500° C. for 6 hours under Ar atmospheric environment. The size of metal nanoparticles inside of nanocup structures can be controlled by adjusting the thickness of a deposited metal film.

Template Removal

Carbon nanostructures inside of AAO templates were released by dissolving the AAO template in 33% hydrofluoric acid solution for deposited nanocups as well as gold inserted nanocup containers.

### B. Results

Nanochannels within an AAO template were formed in 5% oxalic acid with 40-45V for 20-40 sec of anodization. By precisely controlling the anodizing time, the lengths of nanochannels were controlled down to 60 nm for the low aspect ratio nanocup geometry. FIGS. 5A-5C show SEM images of controlled short nanochannels with (a) 70 nm length (anodized at 40V for 25 sec), (b) 200 nm length (anodized at 45V for 35 sec), and (c) 400 nm length (anodized at 40V for 120 sec). The graph in FIG. 5D shows the length of nanochannels as a function of second anodizing time at 45V.

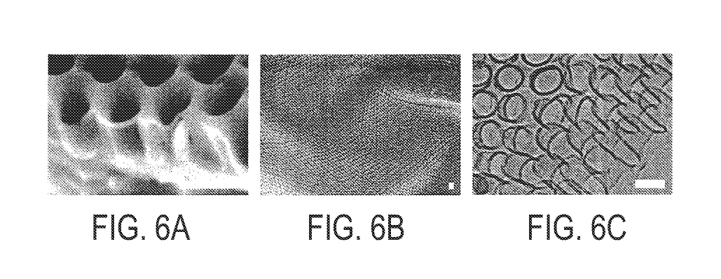

FIGS. 6(A) and 6(B) show (a) SEM images of nanocups connected with a graphitic layer. Polycrystalline graphitic carbon was deposited on both inner and outer surface of AAO nanochannels. FIG. 6B is a low magnification SEM image showing a two dimensional planar nanocup based structure (bottom view). Due to the flexible nature of the two dimensional nanocup-based film, the top, tilted, and side views of connected nanocups can be seen in the TEM image (FIG. 6(C)).

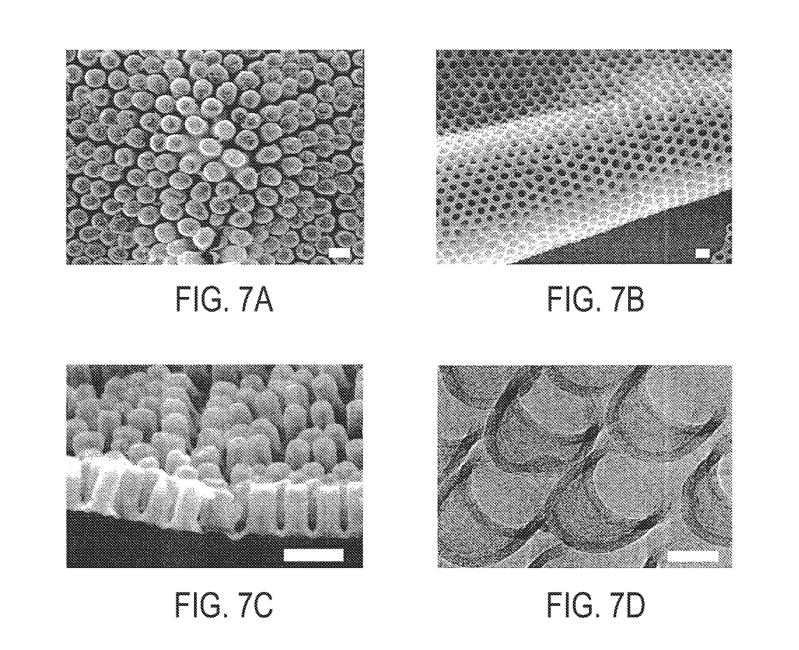

FIGS. 7(A)-7(D) show the scanning electron microscopy (SEM) and transmission electron microscopy (TEM) images of carbon nanocup arrays, connected with a continuous graphitic layer that holds them together. This film of connected nanocups produced has three remarkable features in their structure and morphology. As shown in FIG. 7(A), a two-dimensional graphitic film with highly porous surface was achieved by connecting the highly dense and ordered arrays of nanocups together. The resulting film of the nanocup arrays was flexible, and remained intact even under strong physical deformation, as shown in FIG. 7(B). Through the control of nanopore dimensions (diameter and length), the geometry and structure of the nanocups, such as length, diameter, L/D aspect ratio, and their wall thickness, can be precisely designed and controlled. FIG. 7(C) is a representative high magnification SEM image of a nanocup array, arranged in a highly ordered fashion with 100 nm diameter and 200 nm length. The TEM image of arrays of connected nanocups (80 nm diameter and 80 nm length), shows the formation of nanoscale cup geometry as well as a connected nanocup structure with 10 nm wall thicknesses (FIG. 7(D)). The image also shows the flexibility of the two dimensional nanocup array films with a polycrystalline and disordered graphitic structure of their lattice.

To synthesize individual nanocup and nanoring units with full control over their L/D aspect ratio, Ar ion milling (300 V accelerating voltage and 55 mA emission current) was conducted on the connected arrays of nanocup film deposited in the AAO templates. Striking changes in the structure and morphology of the nanocup films were observed during Ar ion irradiation, as shown in FIG. 8. After about 70 sec of Ar ion irradiation on the two-dimensional nanocup films, etching of a graphitic layer connecting the arrays of individual nanocups occurred and resulted in individually separated nanocup structures. Different lengths of nanocups were obtained by controlling the ion milling time used for etching of the preformed nanocup films.

FIGS. 8(A) and 8(B) are SEM images of highly dense and completely separated nanocups with controlled L/D aspect ratio of 3 and 1, respectively. As the ion irradiation time increased beyond 70 sec, the etching of the bottom graphitic layer of carbon nanocups was initiated, resulting in nanoscale tubular ring morphology. FIGS. 8(C) and 8(D) are SEM images of multilayered and individually separated graphitic nanoring structures, respectively. The second layer of the nanoring arrays (FIG. 8(C)), as well as the top surface of the supporting AAO templates (FIG. 8(D)), are observed through the nanoscale pores originating from the hollow ring morphology of nanorings formed by the Ar ion milling process. Detailed information on the morphological changes, from the connected nanocup films to the separated nanocup and nanoring structures, were visualized using TEM. FIG. 8(E) shows side and top views of separated nanocups. Graphitic carbon layers connecting individual nanocup structure were removed by the Ar ion-milling process. A high resolution TEM image of carbon nanorings (FIG. 8(F)) indicates that the bottom layers of carbon nanocups were completely etched, forming the ring geometry. Energetic Ar ions, travelling parallel to the short tubular axis of nanocups, etch the curved bottom layer of the nanocups by the Ar ion irradiation.

Elucidation of Structure

To elucidate the lattice structure and graphitization of the nanocup and nanoring structures, Raman spectroscopy was performed using a 532 nm laser excitation in the spectral range of 1200 cm⁻¹-1700 cm⁻¹, in which the G band was observed (ascribed to tangential modes of the graphene structure, and the disorder-induced D band, activated by the presence of defects). FIGS. 8(G) and 8(H) show the result of Raman spectra observed from typical multiwalled carbon nanotubes (MWNTs), our nanocup, and nanoring structures of similar diameter. The peak intensity ratio (I_{D}/I_{G}) doubled as the structure changed from long MWNT (10 μm length) to short nanoring structures (40 nm length). This result indicated a higher degree of disorder in the nanoring structures due to the Ar ion irradiation and lower L/D aspect ratio. Another noticeable change in Raman spectra was the blue-shift (up-shift) of G band peak position (FIG. 8(G)). For MWNT and carbon nanocup structures, the usual G band was observed around 1600 cm. However, G band spectra of nanorings with 60 nm and 40 nm lengths were recorded at 1607 cm⁻¹and 1612 cm⁻¹, respectively. Such blue-shift of G band modes in the nanoring structures can be caused by the increase of disorder in the graphitic lattice and possibly higher fractions of sp³ hybridized carbon defect sites due to the ion irradiation.

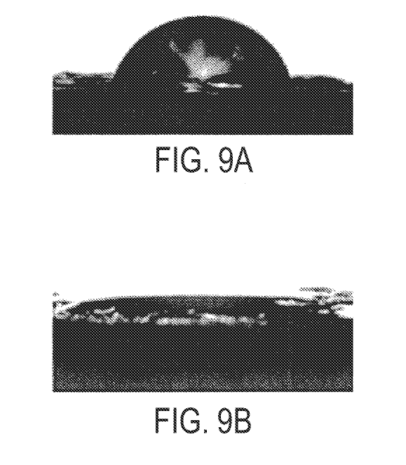

To understand the engineered graphitic nanostructures further, the contact angle change of deionized water on a continuous nanocup film were measured before (FIG. 9A) and after (FIG. 9B) the Ar ion milling process. As shown in FIG. 9A, the contact angles of water droplet on the connected nanocup film ranged from 69° to 73°, indicating the hydrophobicity of nanocup structures. However, after Ar ion irradiation on the connected carbon nanocup film, the contact angles between surface of engineered graphitic nanostructures and the deionized water droplet were dramatically reduced to 0°, showing a hydrophilic property (FIG. 9B). This may indicate a high density of disorder and nanocrystallized graphitic lattice formed from the Ar ion irradiation.

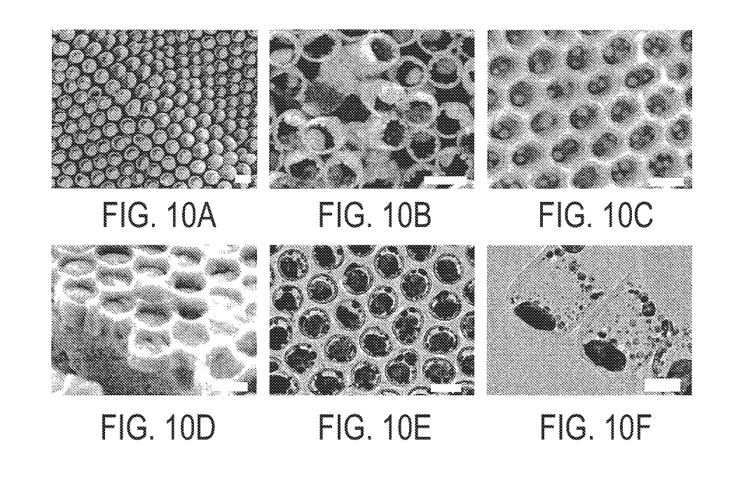

The nanocup structures described herein can be used as container systems at the nanoscale. This was demonstrated by inserting various metals inside the nanocups using an e-beam evaporation, followed by a thermal annealing process under an Ar environment. FIG. 10(A)-10(F) show a collection of diverse carbon nanocup structures holding gold and lead inside. During the annealing process, the deposited metal inside the nanocups was thermally re-evaporated into small metal nanoparticles seen inside the nanocups. As shown in FIG. 10C and 10D, the sizes of inserted metal nanoparticles were controlled by controlling the thickness of a metal film deposited. In the SEM images, gold and lead nanoparticles inside the nanocups were easily observed, as they were visible through the thin graphitic walls. TEM images show that gold nanoparticles were formed only within the pores of connected and isolated nanocups (FIGS. 10(E)-10(F)), resulting in unique hetero-architectures of carbon-metal materials. Using similar methods, silica and polystyrene latex (PSL) were also inserted into carbon nanocups.

### EXAMPLE 2

### Low-Aspect Ratio Carbon Nanocups for Hydrogen Storage

An array of nanocups associated with an AAO template is fabricated as described in Example 1. Nanocups are then subjected to hydrogenation using a furnace connected with high purity H₂ and Ar gas cylinders. Nanocups are heated at temperature below 873K under a mixture of H₂ and Ar atmosphere to avoid recrystallization of the nanocups at high temperature as well as melting of Al substrates.

To tune H₂ storage capability of carbon nanocups, the morphology and structure of carbon nanocups are controlled. To increase hydrogen storage capacity on nanocups, Ar ion-milling or oxygen plasma treatment is applied for tailoring the edge and surface structure of carbon nanocups. The number of graphitic layers in nanocups is increased to enhances the hydrogen adsorption capacity on carbon nanocups.

To increase hydrogen storage, carbon nanocup-catalyst nanoclusters are made by ultra-high vacuum sputter deposition or electrochemical deposition of Pt metal onto an array of nanocups. The distribution of such catalytic metal nanoclusters on carbon nanocups is characterized by SEM and TEM.

### Equivalents

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A hollow, low-aspect ratio nanostructure having a length, a transverse diameter, and a continuous lateral wall defining an interior space, the nanostructure having a ratio of length to transverse diameter of about 0.5 to about 10.

2. The nanostructure of claim 1, wherein the length is about 30 nm to about 1,000 nm.

3. The nanostructure of claim 1, wherein the transverse diameter is about 10 nm to about 500 nm.

4. The nanostructure of claim 1, which is an annular nanostructure having an open top and a closed bottom.

5. The nanostructure of claim 4, having an open top and an open bottom.

6. The nanostructure of claim 5, having a closed top and a closed bottom.

7. The nanostructure of claim 1, comprising carbon or silica.

8. The nanostructure of claim 1, wherein the lateral wall has a thickness of about 5 nm to about 200 nm.

9. The nanostructure of claim 1, wherein the lateral wall defines an interior space of about 30 nm³ to about 0.25 μm³.

10. The nanostructure of claim 1, wherein the nanostructure further comprises an agent within the interior space.

11. An array comprising a plurality of the nanostructures of claim 1.

12. The array of claim 11, wherein the array comprises a support layer that contacts the lateral walls of adjacent nanostructures.

13. The array of claim 12, wherein the support layer is flexible.

14. The array of claim 12, wherein the support layer comprises a graphite layer.

15. The array of claim 14, wherein the graphite layer has a thickness of about 5 nm to about 200 nm.

16. The array of claim 11, wherein the lengths of the nanostructures are uniform.

17. The array of claim 11, wherein the transverse diameters of the nanostructures are uniform.

18. The array of claim 11, wherein about 60% to about 100% of the nanostructures have the same length and/or transverse diameter.

19. A method of forming an array of a plurality of hollow, low-aspect ratio nanostructures, the method comprising:
preparing an anodized aluminum oxide (AAO) template comprising a plurality of nanochannels by two-step anodization; and
disposing a graphitic carbon layer onto the AAO template, thereby producing an array of hollow, low-aspect ratio nanostructures.

20. The method of claim 19, wherein the graphitic carbon layer is disposed onto the AAO template by chemical vapor deposition.

21. The method of claim 19, wherein each nanochannel has a length, a transverse diameter, and a lateral wall defining an interior space, the nanochannel having a ratio of length to transverse diameter of about 1 to about 10.

22. The method of claim 21, further comprising placing an agent into the interior space of one or more of the nanostructures.

23. The method of claim 22, further comprising sealing the nanostructure.

24. The method of claim 19, further comprising dissolving the template to remove the array from the template.

25. A method of forming a hollow, low-aspect ratio nanostructure, the method comprising:
preparing an anodized aluminum oxide (AAO) template comprising a plurality of nanochannels by two-step anodization;
disposing a graphitic carbon layer onto the AAO template to form an array of hollow, low-aspect ratio nanostructures;
removing the array from the template; and
separating one or more nanostructures from the array.

26. The method of claim 25, wherein the separating step comprises inert gas ion milling.

27. A method of delivering a therapeutic or detection agent to a target cell, the method comprising:
providing the nanostructure of claim 1;
modifying the nanostructure with the therapeutic or detection agent; and
administering the modified nanostructure to a subject, thereby delivering the agent to the target cell.

28. The method of claim 27, further comprising linking a targeting agent to the nanostructure.

29. The nanostructure of claim 10, wherein the agent is hydrogen.
